(19) United States
(12) Patent Application Publication
(10) Pub. No.: US 2014/0323341 A1
(43) Pub. Date: Oct. 30, 2014

(54) SYSTEMS AND METHODS FOR ASSESSMENT OF BIOSIMILARITY

(71) Applicants: Sergei S. Makarov, Morrisville, NC (US); Alexander Vladimirovich Medvedev, Durham, NC (US)

(72) Inventors: Sergei S. Makarov, Morrisville, NC (US); Alexander Vladimirovich Medvedev, Durham, NC (US)

(73) Assignee: ATTAGENE, INC., Research Triangle Park, NC (US)

(21) Appl. No.: 14/343,847

(22) PCT Filed: Sep. 8, 2012
(86) PCT No.: PCT/US12/54338

§ 371 Date: Jun. 5, 2014

Related U.S. Application Data
(60) Provisional application No. 61/532,171, filed on Sep. 8, 2011.

(51) Int. Cl. G01N 33/50; G01N 33/68

(52) U.S. Cl. CPC G01N 33/5023; G01N 33/6872
USPC 506/9; 506/18; 506/16

(57) ABSTRACT

A method of determining biosimilarity of a sample composition to a reference composition, including: exposing a test cell system to a sample composition so that the test cell system responds to the sample composition by change in transcription factor activity in the test cell system; generating from the test cell system response an output correlative to the change of transcription factor activity in the test cell system; and determining from comparison of said output with a transcription factor activity reference standard for the reference composition, the biosimilarity of the sample composition to the reference composition. Computer systems and kits for carrying out the determination of biosimilarity of compositions are also described, in which biosimilarity of compositions ranging from simple molecules to complex mixtures can be readily and accurately determined by transcription factor activity analysis.

### CROSS-REFERENCE TO RELATED APPLICATION

The benefit under 35 U.S.C. 119 of U.S. Provisional Patent Application No. 61/532,171 filed Sep. 8, 2011 in the name of Sergei S. Makarov for “SYSTEMS AND METHODS FOR ASSESSMENT OF BIOSIMILARITY” is hereby claimed. The disclosure of such U.S. provisional application is hereby incorporated herein by reference in its entirety, for all purposes.

### FIELD

The present disclosure relates to determination of biosimilarity and to systems and methods for obtaining such determinations

### DESCRIPTION OF THE RELATED ART

In many fields of endeavor, qualitative and quantitative determinations of biosimilarity of compositions are important. Illustrative examples include the fields of environmental toxicology, medicinal chemistry, forensic pathology, law enforcement relating to controlled substances, epidemiology, nutritional counseling, pharmaceutical manufacturing and agricultural crop science, to name only a few.

Despite its importance, the assessment of biosimilarity has resisted efforts to relate biological response to compositional characteristics in a straightforwardly reproducible and meaningful manner.

For instance, in the biopharmaceutical products industry, the molecular complexity of therapeutic biologics, in relation to small molecule drugs, has generated concerns that even putatively identical biologics may nonetheless markedly differ from one another in biological effectiveness and side effects. Such differences may derive from minute variations in bioprocessing conditions used to manufacture the therapeutic biological product, mutational changes in microbial cultures utilized for expression of the biological product, etc. This in turn has impeded the development and regulatory approval of biological products, and subsequent versions of previously approved therapeutic biologics.

Genomic, proteomic, and metabolomic approaches have not been availing to provide practical tools for overcoming these difficulties.

There is accordingly a continuing compelling need for systems and methods to determine biosimilarity in a ready, reproducible and accurate manner.

### SUMMARY

The present disclosure relates to determination of biosimilarity of different compositions. More specifically, the disclosure relates to systems and corresponding methods for determining biosimilarity of compositions based on transcription factor signatures elicited by such compositions in interaction with biosensors.

In one aspect, the invention relates to a method of determining biosimilarity of a sample composition to a reference composition, comprising:

exposing a test cell system to a sample composition so that the test cell system responds to the sample composition by change in transcription factor activity in said test cell system;

generating from the test cell system response an output correlative to the change of transcription factor activity in said test cell system; and

determining from comparison of said output with a transcription factor activity reference standard for the reference composition, the biosimilarity of the sample composition to the reference composition.

In another aspect, the invention relates to a method of determining biosimilarity of compounds, comprising quantifying impacts of said compounds on activities of multiple transcription factors in a test cell system.

A further aspect of the disclosure relates to a method of determining biosimilarity of different compositions, comprising:

exposing each different composition to a corresponding biosensor comprising multiple transcription factors, wherein the corresponding biosensor is adapted to manifest a transcription factor signature in response to the exposure; and

comparing transcription factor signatures of the corresponding biosensors, or of their expression products, to determine biosimilarity of the different compositions in relation to one another.

Another aspect of the disclosure relates to a method of determining biosimilarity of different compositions, comprising:

introducing into a test cell system comprising a multiplicity of transcription factors, a plurality of reporter constructs whose promotors are regulated by the transcription factors;

exposing the test cell system to the different compositions to induce corresponding changes in activities of said multiplicity of transcription factors; and

determining biosimilarity of the different compositions from a plurality of reporter transcripts produced by the reporter constructs and/or a plurality of reporter proteins produced by the reporter constructs in response to the changes in activities of said multiplicity of transcription factors upon exposing the test cell system to the different compositions.

The disclosure in a further aspect relates to an apparatus for determining biosimilarity of different compositions, comprising a computer system adapted to carry out an operation of a method according to the disclosure.

Another aspect of the disclosure relates to a biosimilarity assessment system, including a central facility including a relational database operatively coupled with a server communicationally connected to one or more remote facilities, wherein said one or more remote facilities are arranged for collection and processing of local sample compositions to generate transcription factor activity profiles therefor, and to transmit same to the server, wherein the relational database contains transcription factor activity profiles of reference compositions accessible by the server, and wherein the server is configured to determine from local sample composition transcription factor activity profiles transmitted to it, in relation to transcription factor activity profile(s) of reference compositions accessed from the relational database, the biosimilarity of a local sample composition to a reference composition.

In a further aspect, the invention relates to a kit for carrying out biosimilarity determinations of compositions, comprising transcription factor signatures for reference library compositions in a graphical format, for threshold visual determinations of biosimilarity of a transcription factor signature to reference library signatures.

Another aspect of the disclosure relates to a kit for carrying out biosimilarity determinations of compositions, comprising biosensors, contacting containers in which cells may be contacted with compositions of interest, and instructional documents containing protocols for conducting the contacting operation, and the further processing of the contacted cell samples for analysis of transcription factor signatures.

Other aspects, features and embodiments of the invention will be more fully apparent from the ensuing disclosure and appended claims.

### BRIEF DESCRIPTION OF THE DRAWINGS

FIG. 1 is a schematic representation of an illustrative biosimilarity assessment system 10, according to one embodiment of the present disclosure.

FIG. 2 is a polar coordinate graphical representation of capillary electrophoresis data showing relative fluorescent values of reporter peaks indicative of transcription factor activity, as a function of transcription factor species, in which the transcription factor activity is the activity generated by interaction of a composition of interest with a host cell containing reporter transcription factor units for each of the transcription factor species shown on the graph.

FIG. 3 is a schematic depiction of the process of generating a transcription factor signature for a host cell biosensor in exposure to an evaluated substance.

FIGS. 4 and 5 are transcription factor signatures for two compounds, Substance X and Substance Y, which are used to assess biosimilarity of these respective compounds by comparing imprints of these compounds on activities of multiple transcription factors in biosensor test cells.

FIG. 6 is a logarithmic polar plot of the transcription factor signatures for four organotin compounds, azocyclotin (Azo), cyhexatin (CYH), tributyltin (TBT) and triphenyltin (TPT) at 0.1 mM concentration in exposure to HepG2 cells, with profiles of the cis-FACTORIAL™ activities determined as a fold of induction values versus vehicle-treated control cells, and with the radar graph showing fold-induction data plotted in logarithmic scale.

FIGS. 7-11 show the profiles of transcription factor activity of the sunscreen formulations SB1, SB2, SB3, SB4, and SB5, respectively, at 100 μg/ml in HepG2 cells that were transiently transfected with the optimized cis-FACTORIAL™ library.

FIGS. 12-14 show comparative profiles of transcription factor activity of the sunscreen formulations against one another, at 100 μg/ml in all cases, with FIG. 12 showing superpositioned SB1/SB3/SB5 transcription factor signatures, FIG. 13 showing superpositioned SB1/SB2 transcription factor signatures, and FIG. 14 showing superpositioned SB2/SB4 transcription factor signatures, in HepG2 cells that were transiently transfected with the optimized cis-FACTORIAL™ library.

FIG. 15 is a cluster analysis depiction of the transcription factor signatures of the sunscreen formulations SB1, SB2, SB3, SB4, and SB5, showing that the transcription signatures of biosimilar sunscreen formulations form distinct clusters in unsupervised hierarchical cluster analysis of the transcription factor signatures of the sunscreens and their UV-blocking components.

FIG. 16 is a schematic representation of a biosimilarity assessment system, including a central water quality administration facility, arranged for sample handling and storage, biosensor (test cell) cell culturing, cell plating and processing, database and data analysis, and linked in communication relationship with remote sample input and processing facilities, which may be optionally supplied with reagents, biosensor (test cell) products, and process equipment support from the central facility.

### DETAILED DESCRIPTION

The present disclosure relates to the determination of biosimilarity of different compositions based on transcription factor signatures elicited by such compositions in interaction with biosensors.

Biosimilarity as used herein refers to similarity of biological properties of different compositions, i.e., the similitude of biological response that is produced by different compositions when such different compositions interact with biological systems of interest. The biological systems utilized for such determinations of biosimilarity in accordance with the present disclosure comprise biosensors. Such biosensors comprise cells, which may be present in a variety of forms, including, without limitation, individual cells, cell cultures, single-cell organisms, microbial populations, multicellular organisms, biological specimens taken or derived from such organisms, such as organs, tissue samples, and tissue cultures. The cells may be endogenous cells, exogenously modified cells, or synthetic cells. The cells can be of any suitable types, and can include human cells, animal cells (such as swine cells, rodent cells, canine cells, bovine cells, ovine cells and/or equestrian cells) cloned cells, plant cells, or the like. The cells may be blood cells, cultured cells, biopsied cells, or cells that are fixed with a preservative or bound to a substrate. The cells can be nucleated, such as white blood cells or suspended endothelial cells, or non-nucleated, such as platelets or red blood cells.

As used herein, the singular forms “a”, “an” and “the” are intended to include the plural forms as well, unless the context clearly indicates otherwise.

The various elements, features, aspects, implementations, and embodiments described herein are intended to be non-limitingly construed, and the disclosure therefore is to be understood and interpreted, as encompassing all potential permutations and combinations of such elements, features, aspects, implementations, and embodiments, or a selected one or ones thereof with other elements, features, aspects, implementations, and embodiments, as being within the scope of the disclosure.

The present disclosure enables the assessment of the bioequivalence of compositions, and comparison of their impacts on gene regulatory pathways in a test cell system, in which the test cell system is exposed to compositions to be assessed for bioequivalence, and the profiles of ensuing changes in activities of transcription factors are analyzed to determine the presence or absence and degree of biological similarity of such compositions to one another, wherein one of the profiles may be a reference standard for a reference composition against which a profile of transcription factor activity for the sample composition is assessed.

The disclosure contemplates in one embodiment a method of determining biosimilarity of a sample composition to a reference composition, comprising:

exposing a test cell system to a sample composition so that the test cell system responds to the sample composition by change in transcription factor activity in said test cell system;

generating from the test cell system response an output correlative to the change of transcription factor activity in said test cell system; and

determining from comparison of said output with a transcription factor activity reference standard for the reference composition, the biosimilarity of the sample composition to the reference composition.

In such method, the transcription factor activity reference standard for the reference composition can comprise an averaged transcription factor activity reference for multiple samples of the reference composition.

The test cell system in such method may comprise a synthetic promoter in reporter transcription units. The test cell system can comprise a promoter in reporter transcription units that is responsive to multiple transcription factors. The test cell system can comprise a panel of cells of differing types, wherein each of said cell types responds to exposure to the sample composition by change of transcription factor activity of only one transcription factor that is different in each of the cell types. The test cell system can comprise a panel of cells of differing types, wherein each of said cell types responds to exposure to the sample composition by change of transcription factor activity of multiple transcription factors that are different in each of the cell types.

The present disclosure generally contemplates a variety of different implementations, including implementations in which the change in activities of multiple transcription factors is analyzed by introducing into a test cell system a plurality of reporter constructs whose promotors are regulated by the transcription factors, and the activities of reporter constructs are assessed by analyzing a plurality of reporter transcripts produced by the reporter constructs and/or a plurality of reporter proteins produced by the reporter constructs.

Alternatively, changes of activities of transcription factors can be analyzed by assessing changes in DNA-binding activities in cell extracts, e.g., by using a gel-shift assay.

As a still further alternative, the changes in transcription factor activities can be assessed by analyzing changes in cellular localization of transcription factors (e.g., nuclear translocation of transcription factors) or any other parameters that are associated with transcription factor activities.

The test cell system can be substantially varied in the practice of the present disclosure. For example, the test cell system can comprise an in vitro culture of primary cells or transformed cells or the test cell system can comprise a mixture of different cell types. The test cell system in other embodiments can be or comprise an in vitro organ culture, e.g., a tissue slice culture. In still other implementations, the test cell system can be or comprise organs or tissues of live animals.

In various embodiments, the transcription factor profile can comprise from 2 to 100 transcription factor activities, with lower limits and narrower ranges being useful in specific applications, e.g., from 2 to 50 transcription factor activities, from 2 to 40 transcription factor activities, from 2 to 20 transcription factor activities, or from 2 to 10 50 transcription factor activities.

In algorithmic representations, the transcription factor activity profiles can be represented by vectors with coordinates x1, x2 . . . xN, where xi is the activity of the i^{th} transcription factor, TFᵢ. Comparison of transcription factor profiles can then be carried out by different techniques, e.g., by analyzing correlation of transcription factor profiles, as previously described, or by assessing the Euclidian distance between the transcription factor activity vectors.

Biosensors in the practice of the systems and methods of the present disclosure are utilized to interact with compositions in a manner eliciting a biological response of the biosensor involving transcription factor activity in the cell(s) of the biosensor. The biosensor cells in the methods of the disclosure undergo transformation in response to the compositions with which the cells interact, so that the cells manifest altered transcription factor activity relative to activity in the cell prior to the interaction with the composition of interest. By use of a same biosensor, e.g., respective aliquots of a homogeneous cell population, transcription factor activity profiles (referred to herein as “transcription factor signatures”) attributable to interaction of different compositions with the same biosensor can be compared by any of various suitable comparative techniques to determine biosimilarity of the respective compositions.

Compositions used to selectively challenge, transfect, modify, or otherwise interact with the biosensor can be of any suitable type, and can for example include compounds, chemical complexes, elements, ionic species, multi-component (chemical or material) formulations, cells, ligands, microbes (e.g., viruses, bacteria, fungi, protozoa), nucleic acids, proteins, peptides, antigens, receptors, antibodies, nutrients, minerals, and environmental samples (e.g., air, water, soil), and components, fragments and/or contaminants of the foregoing.

By way of example, biosimilarity of compounds (similarity of their biological properties) can be inferred from the similarity of transcription factor signatures, i.e., by comparing the impacts of the respective compounds on the activities of multiple transcription factors in a test cell system.

For such purpose, the transcription factor signature is generated for each of the compounds to be assessed for biosimilarity. The transcription factor signature can be rendered in any suitable form, including algorithmic, data, and/or graphical forms, as requisite for subsequent analytical comparison, and reporting or other responsive output, using computer(s) and/or networked computer systems that are specially adapted for data acquisition and data processing. Methods of the types described in U.S. Pat. No. 7,771,660 and in U.S. Patent Application Publication 2010009348, and corresponding assays commercially available from Attagene, Inc. (Research Triangle Park, N.C.) under the trademark FACTORIAL can be employed to generate transcription factor signatures in the practice of the present disclosure.

In a specific implementation, the transcription factor signature is generated by constructing a library of reporter transcription units (RTUs), in which each RTU is constructed to include a common plasmid backbone and a unique transcription factor-inducible promoter that is fused to a transcribed reporter sequence. When co-introduced into a cell of interest, e.g., a HepG2 cell, the RTUs produce reporter RNAs in amounts that are commensurate with the activities of the corresponding transcription factors present in the cell. In order to provide equal detection opportunities for different transcription factors, all RTUs are supplied with essentially identical reporter sequences. To distinguish reporter sequences produced by different RTUs, each sequence is provided with a short processing tag, e.g., a Hpa1 restriction cleavage site, the position which varies among the RTUs.

By such arrangement, reporter sequences can be discriminated upon cleavage with a corresponding processing enzyme. The cleaved reporter species subsequent to such enzymatic processing are separated by high resolution capillary electrophoresis (sequencing) and quantified. All operational steps of this detection protocol can be performed using a homogeneous set of reagents in a single reaction tube, thereby providing highly uniform conditions for detecting multiple transcription factors.

Multiplexed detection of transcription factors can be carried out by introducing the transcription factor reporter library into cells of interest using standard transfection techniques. After transfection, total cellular RNA is isolated and submitted to reverse transcription. Reporter cDNAs are amplified by polymerase chain reaction (PCR) using a pair of primers that is common for all reporters. PCR products then are labeled with fluorescent dye and digested by the Hpa1 restriction enzyme, producing a distribution of fluorescently labeled DNA fragments of different lengths that are resolved by capillary electrophoresis (CE) and detected as separate fluorescent peaks.

The capillary electrophoresis data can be processed in any suitable manner to provide an output fluorescence spectrum of reporter peaks that is amenable to further analysis and output using standard spreadsheet computer programs. In one embodiment, the capillary electrophoresis data are processed using Attagraph™ CE processing software (Attagene, Inc., Research Triangle Park, N.C.), to algorithmically subtract background fluorescence and enable precise sizing of reporter peaks and noise/reporter peak discrimination. Fluorescence values of individual peaks may be normalized to the sum of signals of all peaks, to generate standardized data. The resulting relative fluorescence values of the individual reporter peaks, and corresponding RTU activity values, as transcription factor signatures, can then be exported into Microsoft Office® Excel® or other spreadsheet for further analysis, e.g., quantitative comparison of respective transcription factor signatures, to assess biosimilarity of the respective compounds.

In addition to the foregoing illustrative technique, as a methodology enabling the concurrent assessment of activities of many dozens of transcription factors to obtain a transcription factor signature, alternative methods may employ profiling of transcription factors in a panel of multiple parallel reporter cell lines. In general, the number of transcription factors employed to provide the transcription factor signature may be varied, as necessary or desirable for a specific cell or cell line to generate a signature that is sufficient for reliably assessing biosimilarity. Thus, in some instances, the transcription factor signature may be based on the profiling of 50 transcription factors, while in other instances, 5-10 transcription factors may be sufficient. It will therefore be recognized that the number and type of transcription factors may be substantially varied in the broad practice of the present disclosure, from a single transcription factor to 50 or more transcription factors.

The generalized method of the present disclosure can be practiced to assess biosimilarity of two compounds in a simple and reproducible manner, by obtaining a transcription factor signature of one compound in test cells and obtaining a transcription factor signature of another compound in the same (type) test cells, following which the respective transcription factor signatures can be compared utilizing any appropriate algorithms in a computer-implemented determination process. Illustrative algorithmic determinations may for example employ Pearson methods, Chebyshev correlation coefficients, Euclidean distance techniques, non-parametric methods, etc. For multiple (e.g., three or more) compounds, cluster analysis techniques can be used, in which a computer-implemented cluster analysis comparison is made of the multiple compounds, to determine the compounds of highest biosimilarity as being those that cluster with a specific evaluated or reference compound, within the parametric limits that are set for such referential comparison.

The approach of the present disclosure can be readily applied to the assessment of biosimilarity of compositions ranging from simple molecules to complex mixes of materials (e.g., liquid-solid slurries, multicomponent gas mixtures, liquid solutions, powdered solid formulations, etc.).

Biosimilarity determinations in accordance with the present disclosure can be carried out in computer systems, networks and apparatus that are specially adapted to perform one or more of the actions constituting the biosimilarity assessment methods variously described herein.

The disclosure contemplates data acquisition, data transmission, and data processing apparatus that may be arranged to conduct, or to assist the conduct of, biosimilarity determinations. Accordingly, the biosimilarity determination capability, or components thereof, can be implemented as a computer system or computer program product. Computer program product embodiments include a computer program mechanism embedded or otherwise incorporated in a computer readable storage medium.

Any of the methods, or constituent actions of such methods, as variously described herein, can be embodied as a computer program product. The computer program product can be a CD-ROM, a magnetic disk storage product, or any other computer readable data or program storage product. The software in the computer program product may also be distributed electronically, via the Internet or otherwise, by transmission of a computer data signal (in which the software modules are embedded) on a carrier wave operatively transmitted from a transmission locus device to a receiving locus device.

Computer systems or networks employed to determine biosimilarity of two or more compositions, in accordance with the present disclosure, can include databases and/or memories containing reference transcription factor signatures for various compositions, as a library of signatures against which specific compositions can be compared for same or similar biosensors, to assess biosimilarity of an evaluated composition in relation to one or more compositions for which transcription factors reside in the database. The compositions in the database and/or memory can be of any suitable type for the biosimilarity determination, e.g., biological agents, chemical compounds, prescription drugs, environmental toxins, etc.

The database and/or memory can include any other data or information useful for conducting the bio similarity determination, including cellular data associated with specific transcription factor signatures, protocols for conducting the biosimilarity determination, monitoring data logs for longitudinal studies of biosimilarity development and divergence, trans-acting factor profiles, cis-regulatory element activity profiles, bibliographies of relevant publications, research field contacts, and any other information related to biosimilarity determinations.

Computer systems or networks employed to determine biosimilarity of two or more compositions, in accordance with the present disclosure, can also include data transmission devices, components and capability, e.g., for inputting or transmitting transcription factor data, reference transcription factor signatures, etc. to the system or network, such as to a data acquisition module and/or data processing module thereof, and for outputting or transmitting transcription factor data, signatures and biosimilarity information, for reporting or further processing purposes.

Transmission forms of data and other information in such respect can be tangible or intangible, can be embodied in texts, tables, diagrams, photographs, graphs, charts, emails, images or any other visual forms, can be recorded on a tangible media such as paper, plastic transparency sheets, film, and the like, or embodied in computer readable forms (e.g., electronic, electromagnetic, optical or other signals). Information in computer-readable form can be stored in a computer usable storage medium (e.g., CDs, optical disks, magnetic tapes, digital video discs and the like) or in computer(s) in temporary or permanent computer storage, and may reside in “raw” data (i.e., collected but unanalyzed), partially analyzed, or completed analyzed forms.

In an illustrative computer system for determining biosimilarity in accordance with the present disclosure, the computer system includes a central processing unit, a main non-volatile storage unit such as a hard disk drive for storing software and data, controlled by a storage controller, a system memory such as high speed random-access memory (RAM) for storing system control programs, data and application programs, including programs and data loaded from the non-volatile storage unit, a system memory that may also include read-only memory (ROM), a user-interface including input devices such as a keyboard or voice input interface, a display or other output device, a network interface card for connecting to a wired or wireless communication network such as a LAN, WAN, WLAN, or Internet network, internal bus members for interconnecting components of the system, and a power source for the system.

Operationally, the aforementioned computer can be controlled primarily by an operating system that is executed by the central processing unit (CPU) of the computer system. The operating system can be stored in system memory, which may include a file system for controlling access to the various files and data structures of the computer system, a data structure for storing transcription factor signatures and related data, and a data analysis algorithm module for comparing transcription factor signatures and generating an appertaining biosimilarity determination.

The computer system thus can comprise various software program modules and data structures, in which the data structures can comprise any form of data storage system including, without limitation, a flat ASCII or binary file, an Excel spreadsheet, a relational database (SQL), or an on-line analytical processing (OLAP) database (MDX and/or variants thereof). In particular embodiments, the data structures can each be in the form of one or more databases that include hierarchical structure and/or non-hierarchical structure, as appropriate to the system configuration and operation. The data structures may be single data structures, or they may comprise plural data structures, such as databases, spreadsheets, files, archives, etc., which may be hosted on the same computer or on different computers in a network that may be selectively accessed by a computer user.

The computer system correspondingly can include modules and data structures on one or more remote computers, and can be implemented as web-based system, e.g., in which a data analysis algorithm module and/or other modules reside on a central server that is linked in network communication with a client computer, or alternatively in which such modules reside on a client computer that is linked in network communication with a central server including a database of reference transcription factor signatures that is available to the client computer for computational determination of biosimilarity of a specific composition in relation to reference compositions for which reference transcription factor signatures reside on the central server in a central database library or file. The central server may for example host an interactive webpage linking to or providing computational ability for the data analysis algorithm module.

In one embodiment, the computer system and associated facilities are arranged for biosimilarity determinations in a configuration that includes a detection facility equipped with apparatus and instrumentation for carrying out detection protocols, including RNA isolation, reverse transcription, PCR amplification, fluorescent labeling, restriction enzyme digestion, sample cleanup, capillary electrophoresis, primary data analysis and data storage. The detection facility may be operationally linked with a tissue culture facility that is compliant with biosafety requirements and is adapted to perform cell-based steps of the biosimilarity determination, including cell storage, propagation, cell plating for screening experiments, transfection and exposure to compositions. The detection and tissue culture facilities may in turn be operationally linked to a sample handling and storage facility that performs intake, aliquotting, reformatting and storage of samples received for biosimilarity determinations. Samples processed up a sample handling and storage facility may include stabilized cell lysates, RNA preparations, and chemical compositions. The respective detection, tissue culture and sample handling and storage facilities may be situated in an integrated manner at one geographic location, or alternatively such facilities or selected ones or components thereof may be sited remotely from other(s), as necessary or desirable in a given implementation of the computer system and associated facilities.

The present disclosure also contemplates the provision of various kits as useful for carrying out biosimilarity determinations of compositions.

In one embodiment, the kit comprises transcription factor signatures for reference library compositions in a graphical format, to facilitate threshold visual determinations of biosimilarity of a transcription factor signature to reference library signatures, e.g., as a toxicological or epidemiological tool to quickly rule in or rule out environmental and etiological agents on the basis of perceived visual similarity or dissimilarity of graphical format transcription factor signatures.

In another embodiment, the kit comprises biosensors, e.g., specific cell line cells that have been transfected with reporter transcription units, and contacting containers in which the cells may be contacted with compositions of interest, together with instructional documents containing protocols for conducting the contacting operation, and the further processing of the contacted cell samples for analysis of transcription factor signatures. Kits in other embodiments may comprise, or further comprise, lyzing media, transfection vectors, restriction enzymes, reverse transcription reagents, PCR primers, fluorescent dyes, discs or flash drives containing capillary electrophoresis data processing software, transcription factor signature-generation software, and/or software for conducting other of the component operations in the biosimilarity determination.

In still other embodiments, kits may be constituted with any one or more of the foregoing kit components.

The features and advantages of the systems and methods of the present disclosure are more fully shown with respect to the following non-limiting drawings figures and appertaining description.

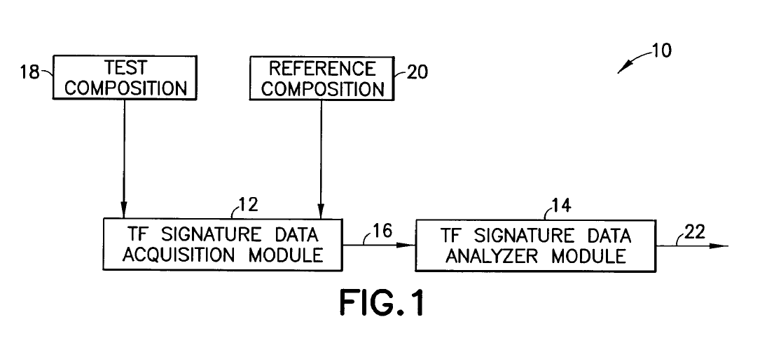

FIG. 1 is a schematic representation of an illustrative biosimilarity assessment system 10, according to one embodiment of the present disclosure. The system 10 includes a transcription factor signature acquisition module 12 operationally linked in information transmission relationship, by information transmission link 16, to transcription factor signature data analyzer module 14 adapted to output information related to the biosimilarity determination for the test composition 18 introduced to the transcription factor signature acquisition module 12. Such output information from transcription factor signature data analyzer module 14 may for example comprise a biosimilarity report for the test composition 18, e.g., in relation to reference composition 20 introduced to the transcription factor signature data acquisition module 12. Alternatively, the output information from the transcription factor signature data analyzer module 14 may comprise a data signal that is further processed to generate a biosimilarity determination, such as by input to a network for communication to a central server adapted to generate such biosimilarity determination, and to communicate such determination back to the transcription factor signature data analyzer module 14 and/or to other devices coupled by wired or wireless connections to the network.

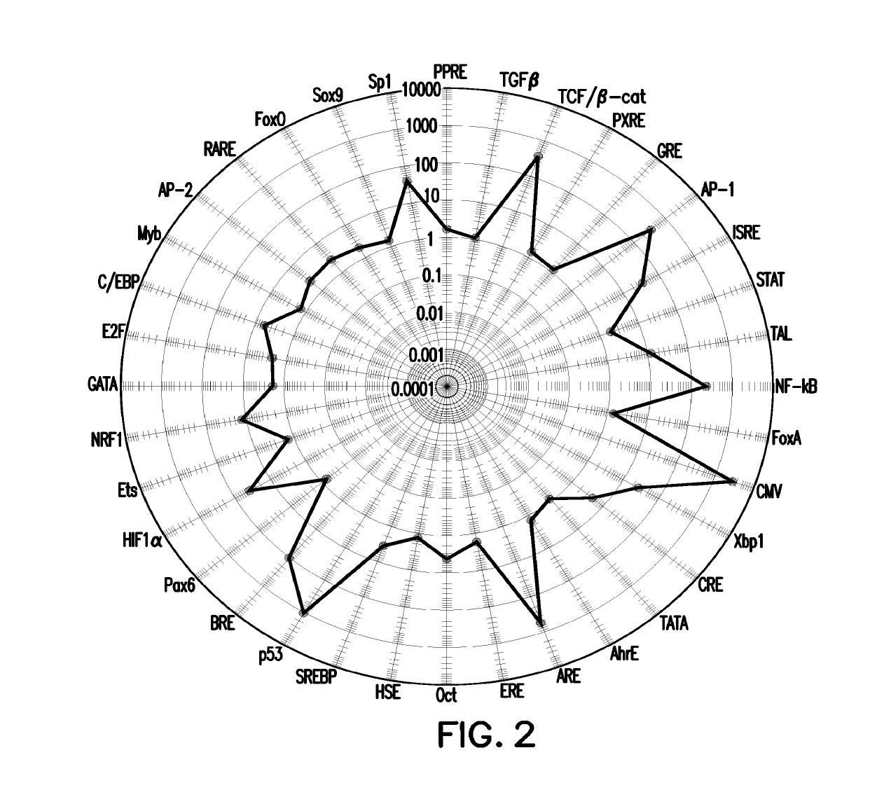

FIG. 2 is a polar coordinate graphical representation of capillary electrophoresis data showing relative fluorescent values of reporter peaks indicative of transcription factor activity, as a function of transcription factor species, in which the transcription factor activity is the activity generated by interaction of a composition of interest with a host cell containing reporter transcription factor units for each of the transcription factor species shown on the graph.

The transcription factor activity graph of FIG. 2 shows the fluorescence peaks of the activated transcription factors as radially outwardly projecting from a baseline circle representing a non-activated state of corresponding transcription factors whose data points appear at the baseline circle, and with the radial extent of the fluorescence peaks being in arbitrary fluorescence units. In this graph, 36 transcription factors have been assayed, and their alphabetic designations appear around the outer periphery of the polar graph.

It is evident from visual inspection of the graph that the transcription factors Sp1, TCF/βcat, AP-1, ISRE, TAL, NF-κB, CMV, Xbp1, CRE, ARE, Oct, SREBP, p53, BRE, HIF-1α, NRF1, and C/EBP show increased activity in relation to the other transcription factors, and that the polar graph provides a “fingerprint” of the interaction of the transcription factor units-containing host cell and the composition of interest.

Accordingly, by comparison of the transcription factor signature on each of respective polar graphs, with respect to peak coordinates specifying a radial distance from the base circle, biosimilarity of the respective compositions used to generate the corresponding graphs can be quantitatively ascertained from the relational congruence of the respective transcription factor signature graphs for such compositions.

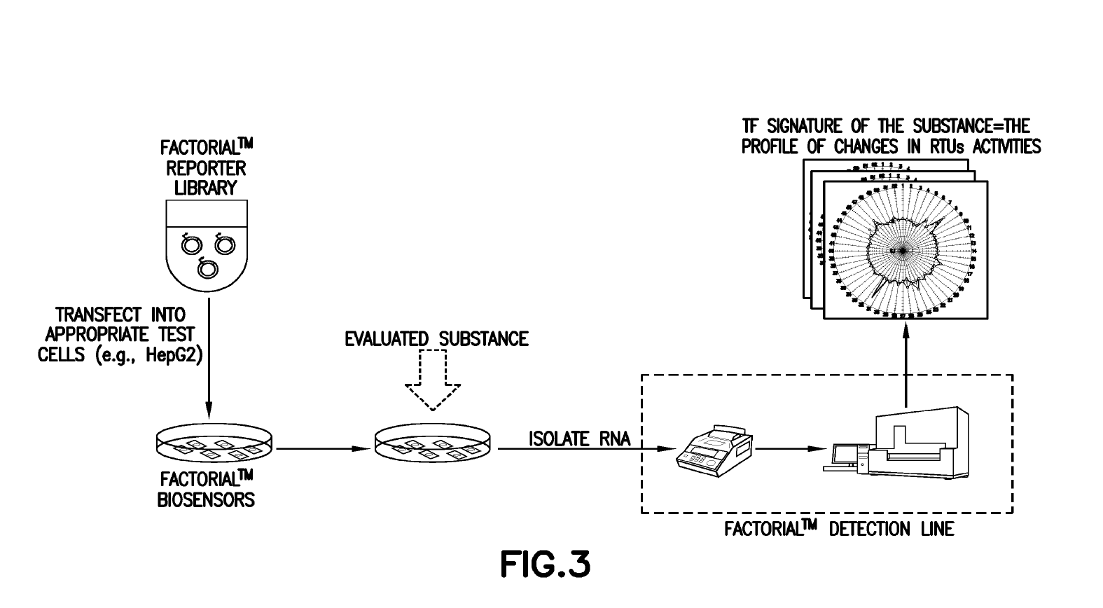

FIG. 3 is a schematic depiction of the process of generating a transcription factor signature for a host cell biosensor in exposure to an evaluated substance. As shown, a reporter library (“FACTORIAL™ reporter Library”) including plasmid constructs incorporating reporter transcription factor units is transfected into appropriate test cells, e.g., HepG2 cells, to form a biosensor cell population (“FACTORIAL™ biosensors”). The biosensor cell population is then exposed to the “evaluated substance,” i.e., the material composition for which the transcription factor signature is to be generated. After exposure to the evaluated substance, reporter RNA produced in the cell by action of the reporter transcription units in the transfecting plasmids is isolated from the exposed biosensor cells. As previously described, the amounts of the respective RNAs are commensurate with the activities of the corresponding transcription factors present in the transfected cell in response to the exposure to the evaluated substance.

The isolated RNAs tagged with variably positioned cleavage site tags are processed by reverse transcription to form reporter cDNAs that are amplified by PCR using a primer pairs that are common for all reporters, following which the PCR products are labeled with fluorescent dye and contacted with restriction enzyme to produce a distribution of fluorescent labeled DNA fragments of different lengths. These different length fragments are resolved by capillary electrophoresis and detected as separate fluorescent peaks (“FACTORIAL™ detection line”).

The transcription factor signature then is generated in polar coordinate graphs of the fluorescent peaks that in the aggregate reflect the profile of changes in activities of the reporter transcription units. In this manner, the biosensors are employed for assessing the imprint of a substance on activities of multiple transcription factors in test cells, i.e., the transcription factor signature.

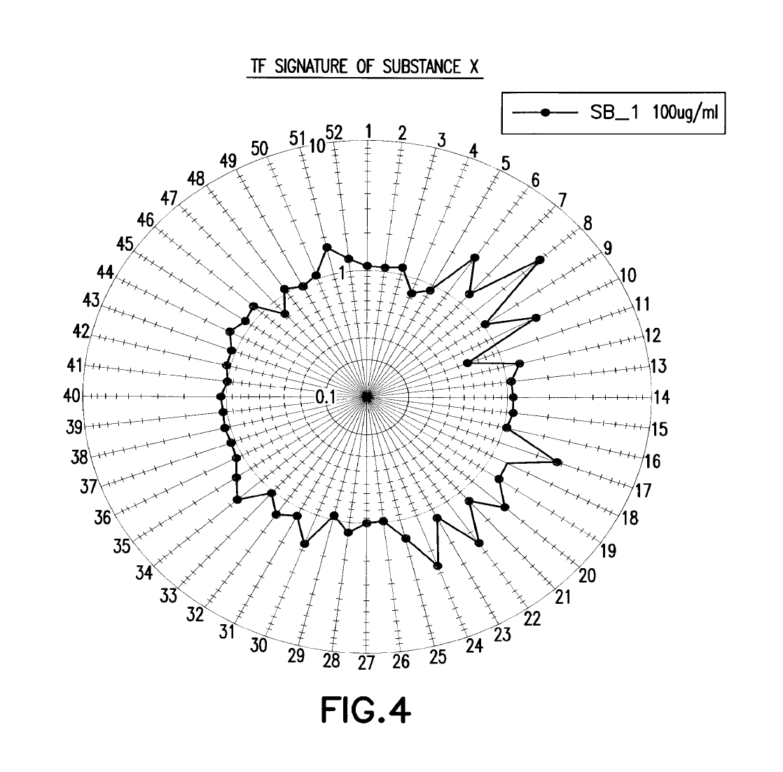

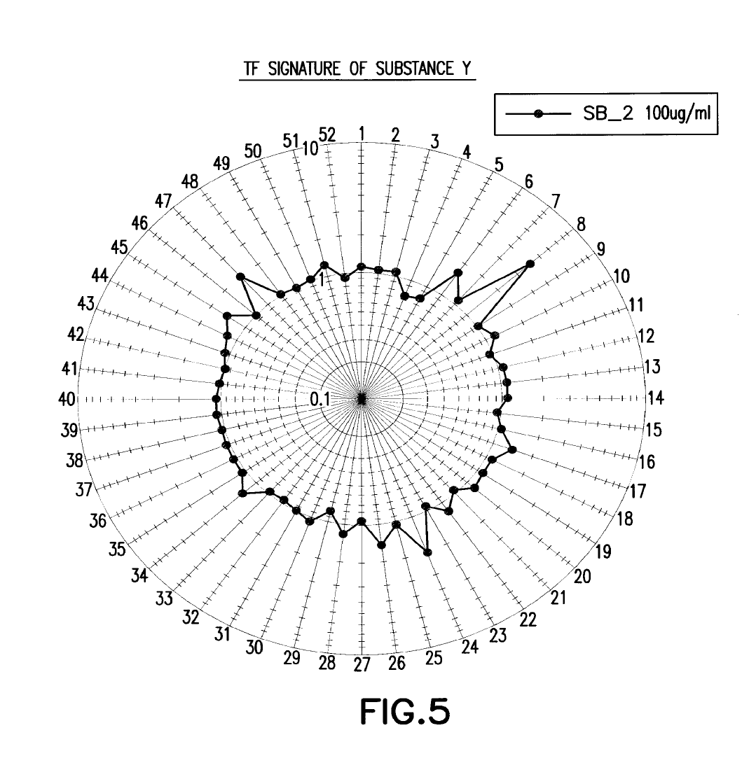

FIGS. 4 and 5 are transcription factor signatures for two compounds, Substance X and Substance Y, which are used to assess biosimilarity of these respective compounds by comparing imprints of these compounds on activities of multiple transcription factors in biosensor test cells.

Using the data from these transcription factor signatures, the Pearson correlation function r of the transcription factor signatures is computed by the following procedure.

The TF signatures of compounds X and Y are a series of n measurements of X and Y written as xi and yi where xi and yi are the fold-change values of the individual reporters, and i=1, 2, . . . , n.

The biosimilarity of the respective compounds can be calculated as a correlation coefficient r between TF signatures of X and Y:

$${T}_{xy} = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2} {\sum }_{i = 1}^{n} {( {y}_{i} - \overline{y} )}^{2}}}$$

where X̅ and Y̅ are the sample means of X and Y.

For fully similar compounds, r=1.0, and for fully dissimilar compounds, r=0.0

Examples 1 and 2 further illustrate the determination of biosimilarity for specific compositions.

### Example 1

Assessing biosimilarities of simple chemical compounds. Organotins.

The following compounds

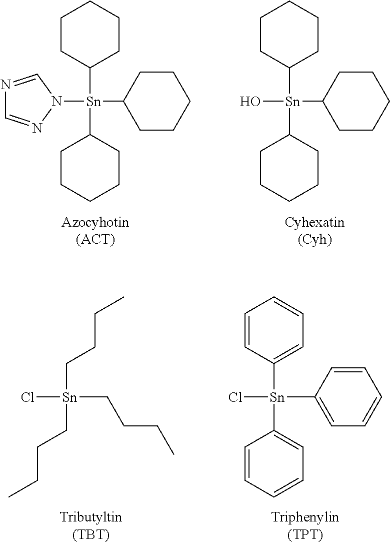 [SMILES: c1nc[n]([Sn]([CH]2CCCCC2)([CH]2CCCCC2)[CH]2CCCCC2)n1 ; [OH][Sn]([CH]1CCCCC1)([CH]1CCCCC1)[CH]1CCCCC1 ; CCC[CH2][Sn]([Cl])([CH2]CCC)[CH2]CCC ; [Cl][Sn]([c]1ccccc1)([c]1ccccc1)[c]1ccccc1]

were assessed for biosimilarity by generating transcription factor profiles of NR activity in HepG2 cells. Cells of the human hepatocellular carcinoma cell line HepG2 were transiently transfected with an optimized trans-FACTORIAL™ library. Twenty four hours after transfection cells were washed and supplied with fresh low serum (1% FBS) culture medium and treated with inducer for 24 hours. The NR signatures of the series of structurally related organotin compounds were generated. The trans-FACTORIAL™ assay was conducted in HepG2 cells treated for 24 hours with azocyclotin (Azo), cyhexatin (CYH), tributyltin (TBT) or triphenyltin (TPT) at 0.1 mM concentration. Profiles of the cis-FACTORIAL™ activities was determined as a fold of induction values versus vehicle-treated control cells. The radar graph shows fold-induction data plotted in logarithmic scale. The NR activities were analyzed as described in Romanov, et al. U.S. Pat. No. 7,700,284. The correlation coefficient between the TF profiles was calculated by Pearson correlation. The correlation coefficient r for the respective compounds is shown in Table 1 below.

TABLE 1
r. correlation between organotins' TF signatures

| | ACT | Cyh | TBT | TPT |
|---|---|---|---|---|
| ACT | 1 | 0.95 | 0.95 | 0.97 |
| Cyh | 0.95 | 1 | 0.93 | 0.95 |
| TBT | 0.95 | 0.93 | 1 | 0.95 |
| TPT | 0.97 | 0.95 | 0.95 | 1 |

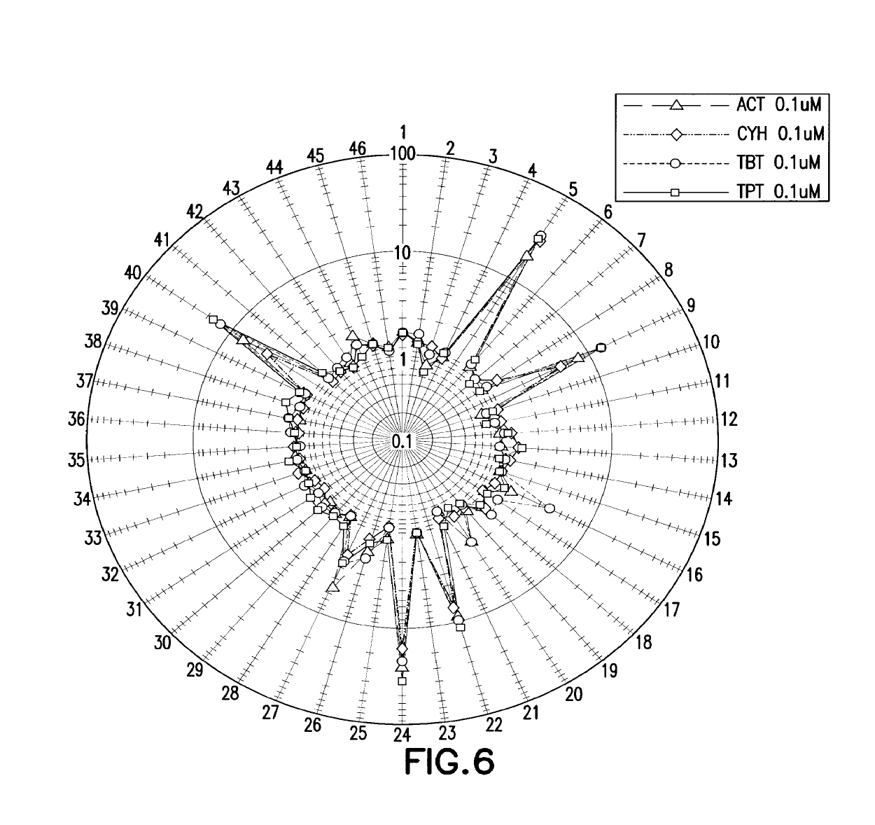

FIG. 6 is a logarithmic polar plot of the transcription factor signatures for the four organotin compounds, azocyclotin (Azo), cyhexatin (CYH), tributyltin (TBT) and triphenyltin (TPT) at 0.1 mM concentration in exposure to HepG2 cells, with profiles of the cis-FACTORIAL™ activities determined as a fold of induction values versus vehicle-treated control cells, and with the radar graph showing fold-induction data plotted in logarithmic scale for 46 transcription factors.

The 46 transcription factors are identified in Table 2 below.

TABLE 2

| # | Name: |
|---|---|
| 1 | FXR |
| 2 | AR |
| 3 | RARg |
| 4 | GAL4 |
| 5 | RXRa |
| 6 | GR |
| 7 | RARb |
| 8 | RARa |
| 9 | PPARg |
| 10 | ERRg |
| 11 | RORb |
| 12 | ERa |
| 13 | LXRa |
| 14 | ERRa |
| 15 | M-19 |
| 16 | PXR |
| 17 | THRa1 |
| 18 | LXRb |
| 19 | CAR |
| 20 | PPARa |
| 21 | RORg |
| 22 | RXRb |
| 23 | HNF4a |
| 24 | NURR1 |
| 25 | VDR |
| 26 | PPARd1 |
| 27 | NUR77 |
| 28 | GCNF |
| 29 | COUP-TF2 |
| 30 | PNR |
| 31 | LRH |
| 32 | Rev-ErbA |
| 33 | MR |
| 34 | COUP-TF1 |
| 35 | TR4 |
| 36 | DAX1 |
| 37 | Rev-Erb8 |
| 38 | RORa |
| 39 | PR |
| 40 | RXRg |
| 41 | SF-1 |
| 42 | SHP |
| 43 | TLX |
| 44 | THRb |
| 45 | EAR2 |
| 46 | TR2 |

The results show TPT and ACT to have the closest biosimilarity to one another among the four compounds.

### Example 2

Biosimilarities of complex chemical compositions, in the form of branded sunscreen formulations, were assessed in this Example. The identification of the respective sunscreen formulations is set out in Table 3 below.

TABLE 3

| ID: | BarCode: | Description |
|---|---|---|
| SB_1 | 041100058133 | Coppertone - Sunscreen Lotion w/Avobenzone |
| | | 50SPF |
| SB_2 | 079656008661 | BB BABY TEAR FREE SPF50 |
| SB_3 | 381371010332 | Aveeno Continuous Protection Sunblock Lotion |
| | | SPF 55, 4 oz |
| SB_4 | 050428168691 | CVS Pharmacy Baby Lotion Sunscreen 60% |
| SB_5 | 086800870203 | Neutrogena Ultimate Sport Sunblock Lotion |
| | | (SPF 70+) |

Active (UV-blocking) ingredients of the respective formulations are set out in Table 4 below.

TABLE 4

| SB_1 | SB_2 | SB_3 | SB_4 | SB_5 |
|---|---|---|---|---|
| Avoenzone | Homosalate | Avobenzone | Titanium | Avobenzone |
| 3% | 15%* | 3% | Dioxide | 3% |
| | | | 4.9% | |
| | | | Zinc | |
| Homosalate | Octinoxate | Homosalate | Oxide | Homosalate |
| 13%* | 7.5% | 10%* | 4.7% | 15%* |
| Octisalate | Octisalate | Octisalate | | Octisalate |
| 5% | 5% | 5% | | 5% |
| Octocrylene | Titantium | Octocrylene | | Octocrylene |
| 7% | Dioxide | 2.8% | | 4% |
| | 2.4% | | | |
| Oxybenzone | | Oxybenzone | | Oxybenzone |
| 4% | | 6% | | 5% |

Sunscreens SB1, SB3, and SB5 are formulations containing the same active ingredients. Each sunscreen also contains a variety of non-essential but biologically active components, as set out in Table 5 below.

TABLE 5

| SB1 | SB2 | SB3 | SB4 | SB5 |
|---|---|---|---|---|
| Aluminum Starch | Aloe Barbadensis (Aloe Vera) | | | Acrylates/C10-30 Alkyl Acrylate |
| Octenylsuccinate | Leaf Juice | Ascorbic Acid | Alumina | Crosspolymer |
| Benzyl Alcohol | Alumina | Behenyl Alcohol | Arachidyl Alcohol | Acrylates/Dimethicone Copolymer |
| | | | | Adipic Acid/Diethylene Glycol/ |
| | | | | Glycerin |
| Carbomer | Butylparaben | BHT | Arachidyl Glycoside | Crosspolymer |
| | C30-38 Olefin/Isopropyl | | | |
| Dimethicone | Maleate/MA Copolymer | Butylene Glycol | Ascorbic acid | Artemia Extract |
| | Cetyl PEG/PPG-10/1 | | | |
| Disodium EDTA | Dimethicone | Butyloctyl Salicylate | Avena Sativa (oat) Kernal Extract | Beeswax |
| Fragrance | Dibutyl Adipate | Caprylyl Methicone | Beeswax | Bisabolol |
| | | Diethylhexyl 2,6- | | |
| Methylparaben | Dimethicone | Naphthalate | Behenyl Alcohol | Cyclopentasiloxane |
| Polyglyceryl-3 | | | | |
| Distearate | Disodium EDTA | Dimethicone | Benzyl Alcohol | Dipotassium Glycyrrhizate |
| Propylparaben | Ethylparaben | Disodium EDTA | BHT | Ethylparaben |
| Sorbitan Isostearate | Glycerin | Ethylhexyl Stearate | Bisabolo | Fragrance |
| Sorbitol | Isobutylparaben | Ethylhexylglycerin | Butylene Glycol | Glycerin |
| Stearic Acid | Isohexadecane | Ethylparaben | Butyloctyl Salicylate | Glyceryl Stearate |
| Tocopherol (Vitamin E) | Methylparaben | Fragrance | Cetyl Dimethicone | Methylisothiazolinone |
| Triethanolamine | Ozokerite | Glyceryl Stearate | Dimethicone | Methylparaben |
| VP/Eicosene | | Glycine Soja | | |
| Copolymer | PEG-8 | (Soybean) Protein | Dimethicone PEG-8 Laurate | Octadecene/MA Copolymer |
| Water | Phenoxylethanol | Methylparaben | Dipotassium Glycyrrhizate | PEG-100 Stearate |
| | Propylparaben | PEG-100 Stearate | Disodium EDTA | Propylparaben |
| | Retinyl Palmitate | Phenoxyethanol | Ehtylhexylglycerin | Saccharomyces/Calcium Ferment |
| | Sodium Ascorbyl Phosphate | Propylparaben | Glycerin | Saccharomyces/Magnesium Ferment |
| | Sodium Chloride | Silica | Glyceryl Stearate | Saccharomyces/Potassium Ferment |
| | | | Hydroxyethyl Acrylate/Sodium | |
| | Sodium Citrate | Sodium Polyacrylate | Acryloyldimethyl Taurate | Saccharomyces/Sodium Ferment |
| | | Styrene/Acrylates | Copolymer | |
| | Tocopheryl Acetate | Copolymer | Isohexadecane | Saccharomyces/Zinc Ferment |
| | (Vitamin E) | | | |
| | Water | Tocopheryl Acetate | Methicone | Silica |
| | | Trideceth-6 | Methylisothiazolinone | Triethanolamine |
| | | Trimethylsiloxysilicate | Panthothenic Acid | Water |
| | | VP/Hexadecene | | |
| | | Copolymer | PEG-100 Stearate | |
| | | Water | PEG-8 | |
| | | Xanthan Gum | Polyaminopropyl Biguanide | |
| | | | Potassium Sorbate | |
| | | | Retinyl Palmitate | |
| | | | Silica + 6 other compounds | |

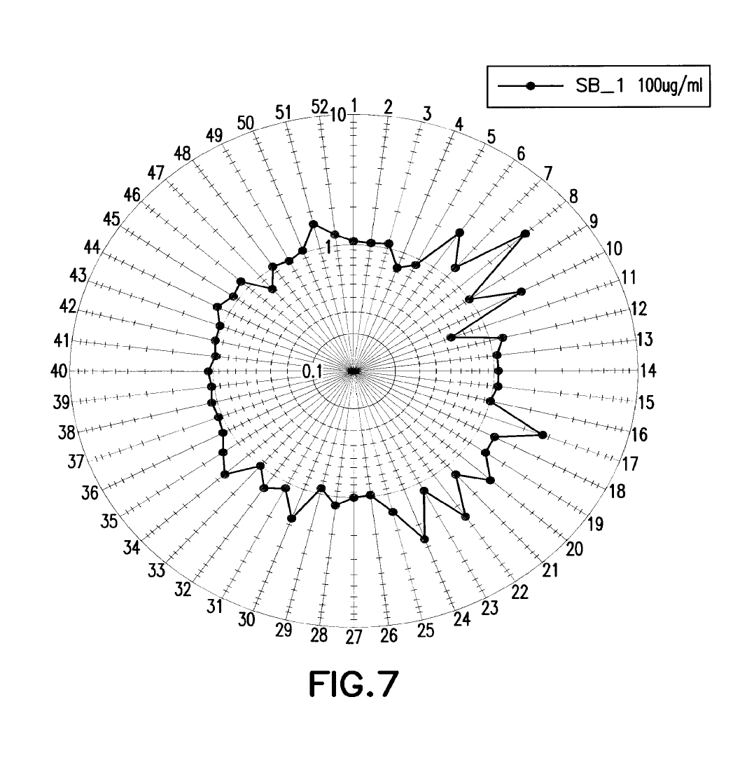

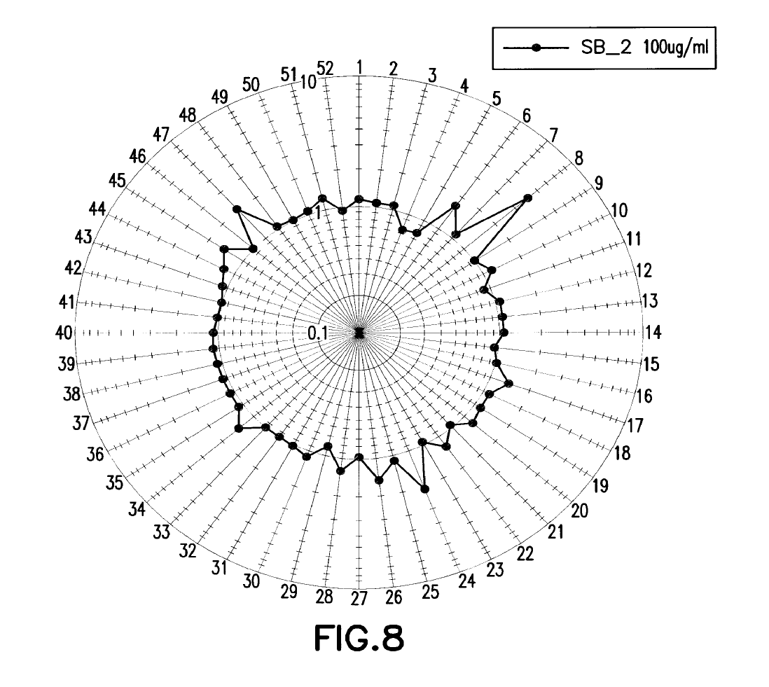

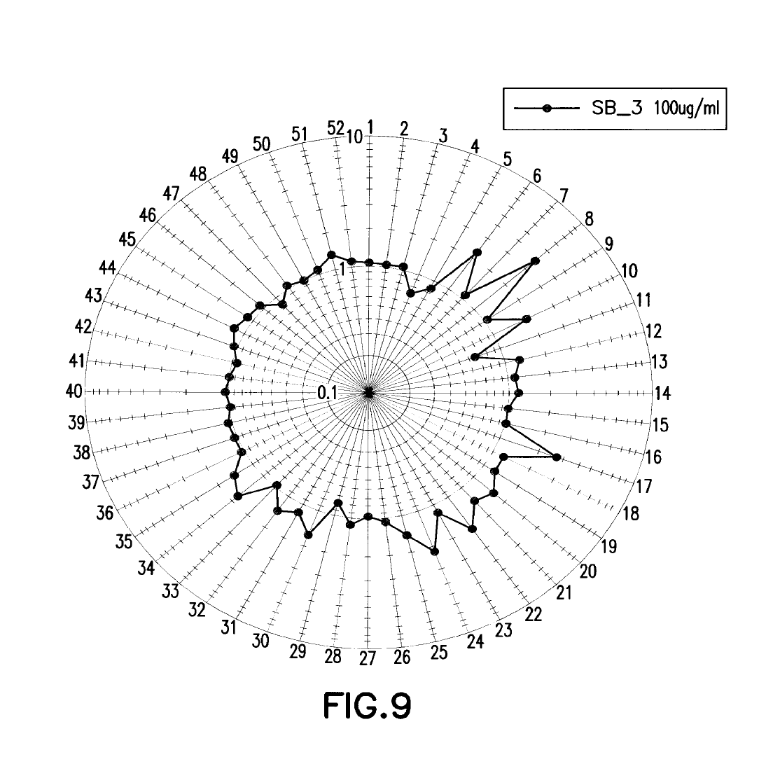

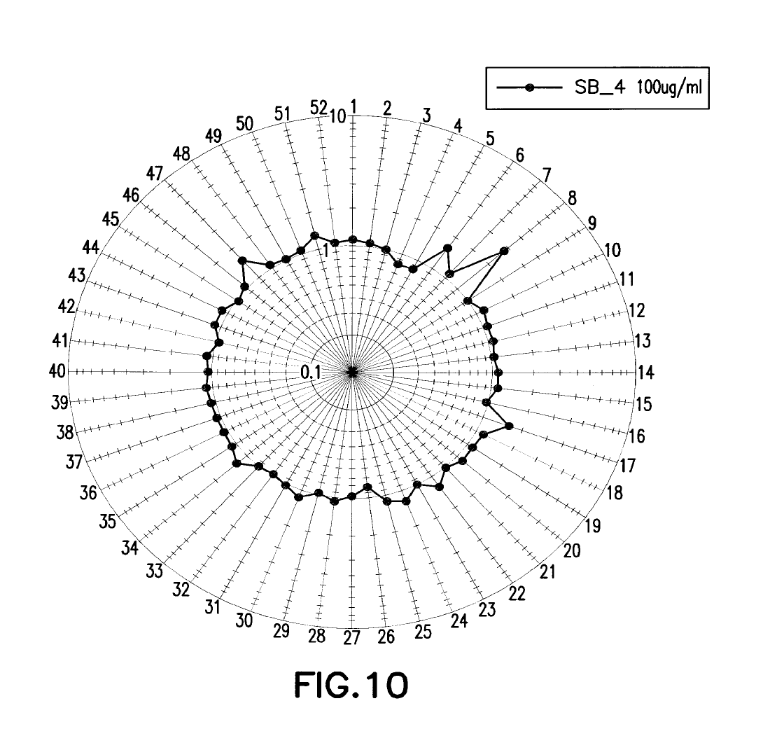

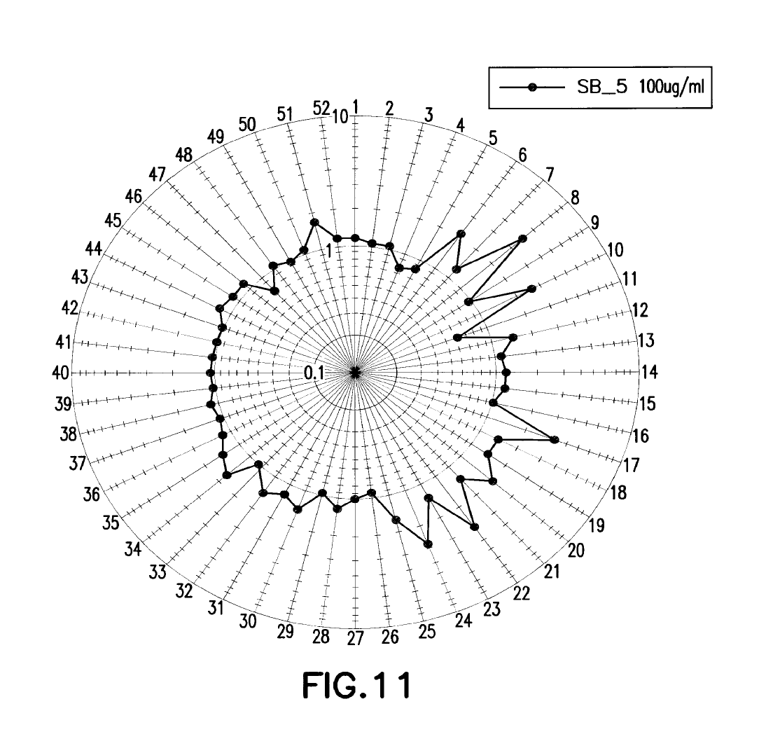

FIGS. 7-11 show the profiles of transcription factor activity of the sunscreen formulations SB1, SB2, SB3, SB4, and SB5, respectively, at 100 μg/ml in HepG2 cells that were transiently transfected with the optimized cis-FACTORIAL™ library.

Twenty four hours after transfection, the cells were washed and supplied with fresh low serum (1% FBS) culture medium and treated with inducer for 24 hours. Profiles of the cis-FACTORIAL™ activities was determined as the fold of induction values versus vehicle-treated control cells. The radar graphs show fold-induction data plotted in logarithmic scale. TF activities were assessed using methods described in Romanov, et al., 2008 (Nature Methods). The 52 transcription factors used in the transcription factor signatures are identified in Table 6 below.

TABLE 6

| # | Name: |
|---|---|
| 1 | TGFb |
| 2 | M-06 |
| 3 | HNF6 |
| 4 | TCF/b-cat |
| 5 | E-Box |
| 6 | PPRE |
| 7 | NFI |
| 8 | PXR |
| 9 | GRE |
| 10 | AP-1 |
| 11 | ISRE |
| 12 | MRE |
| 13 | STAT3 |
| 14 | TAL |
| 15 | NF-kB |
| 16 | FoxA2 |
| 17 | CMV |
| 18 | Xbp1 |
| 19 | CRE |
| 20 | Ahr |
| 21 | EGR |
| 22 | NRF2/ARE |
| 23 | TA |
| 24 | ERE |
| 25 | Oct-MLP |
| 26 | DR4/LXR |
| 27 | HSE |
| 28 | SREBP |
| 29 | p53 |
| 30 | BRE |
| 31 | Pax6 |
| 32 | HIF1a |
| 33 | M-19 |
| 34 | VDRE |
| 35 | RORE |
| 36 | M-32 |
| 37 | Ets |
| 38 | GLI |
| 39 | NRF1 |
| 40 | GATA |
| 41 | E2F |
| 42 | C/EBP |
| 43 | Myb |
| 44 | PBREM |
| 45 | IR1 |
| 46 | AP-2 |
| 47 | DR5 |
| 48 | M-61 |
| 49 | FoxO |
| 50 | Sox |
| 51 | Sp1 |
| 52 | Myc |

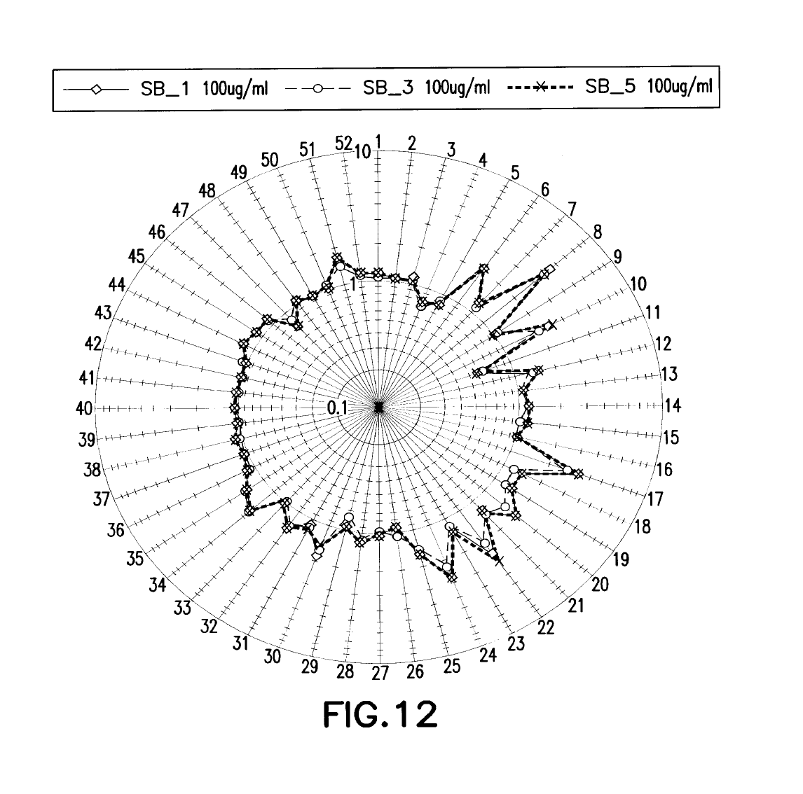

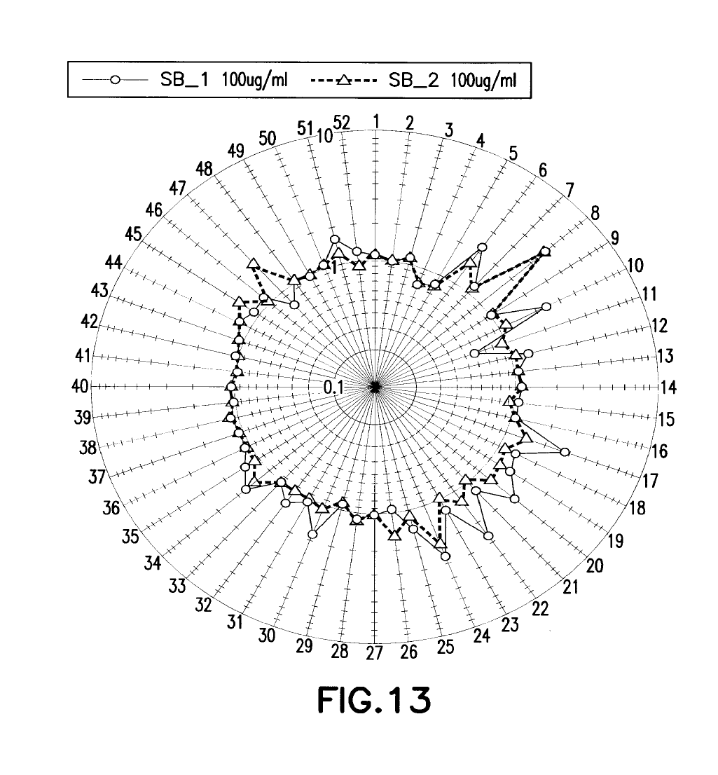

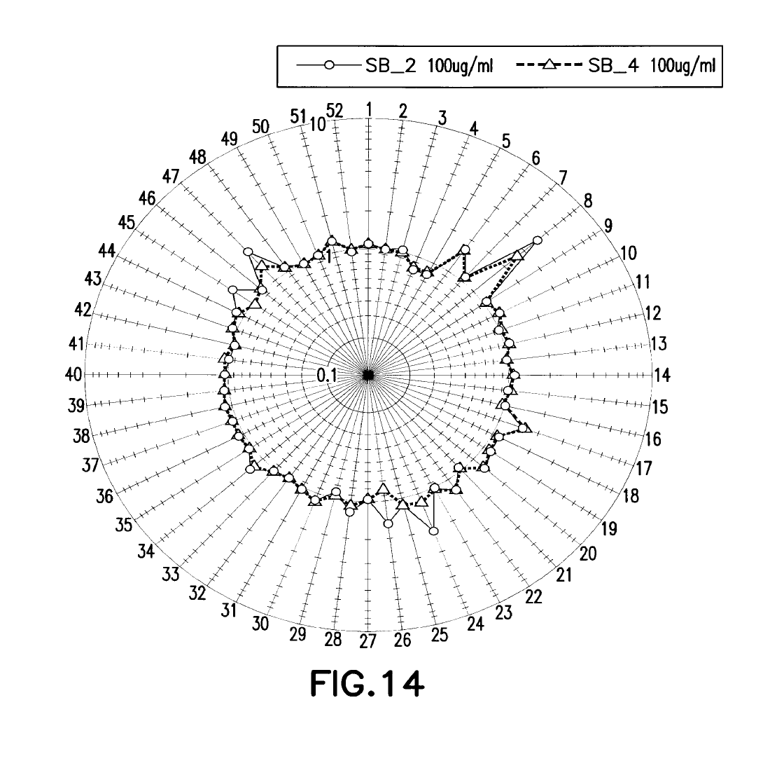

FIGS. 12-14 show comparative profiles of transcription factor activity of the sunscreen formulations against one another, at 100 μg/ml in all cases, with FIG. 12 showing superpositioned SB1/SB3/SB5 transcription factor signatures, FIG. 13 showing superpositioned SB1/SB2 transcription factor signatures, and FIG. 14 showing superpositioned SB2/SB4 transcription factor signatures, in HepG2 cells that were transiently transfected with the optimized cis-FACTORIAL™ library.

Twenty four hours after transfection, the cells were washed and supplied with fresh low serum (1% FBS) culture medium and treated with inducer for 24 hours. Profiles of the cis-FACTORIAL™ activities were determined as the fold of induction values versus vehicle-treated control cells, with the radar graphs showing fold-induction data plotted in logarithmic scale.

The transcription factors were the same as set out above in Table 6. The correlation coefficient r for the respective formulations SB1-SB5 is shown in Table 7 below.

TABLE 7

| | SB_1 | SB_2 | SB_3 | SB_4 | SB_5 |
|---|---|---|---|---|---|
| SB_1 | 1 | 0.65 | 0.97 | 0.74 | 0.98 |
| SB_2 | 0.65 | 1 | 0.70 | 0.80 | 0.59 |
| SB_3 | 0.97 | 0.70 | 1 | 0.78 | 0.96 |
| SB_4 | 0.74 | 0.80 | 0.78 | 1 | 0.72 |
| SB_5 | 0.98 | 0.59 | 0.96 | 0.72 | 1 |

The results show formulations SB1, SB3 and SB5 to have the closest biosimilarity to one another among the five formulations.

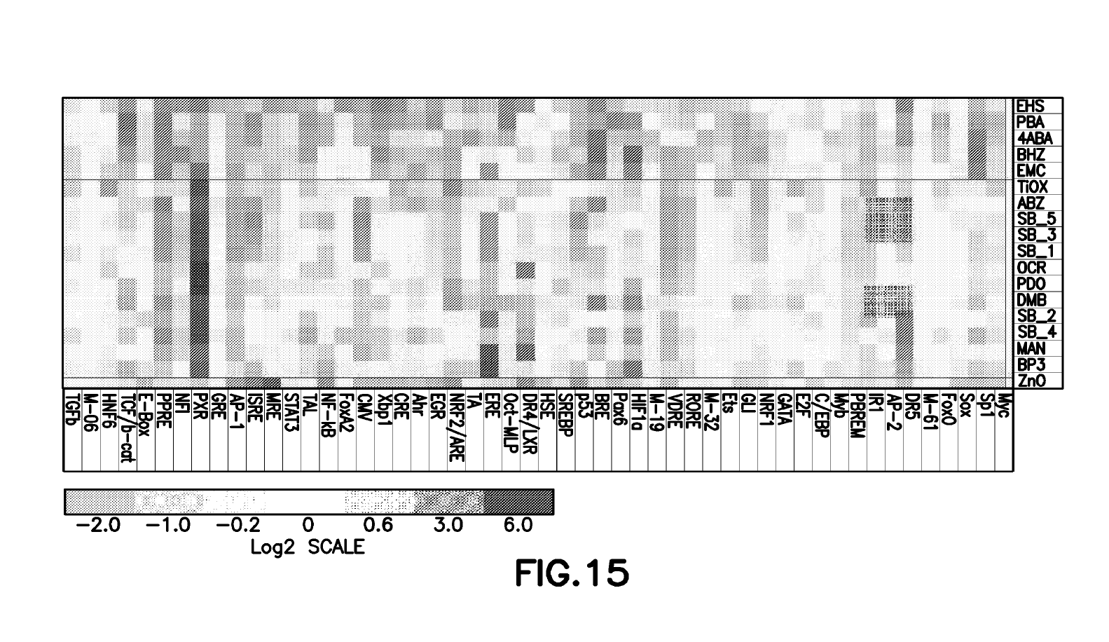

FIG. 15 is a cluster analysis depiction of the transcription factor signatures of the sunscreen formulations SB1, SB2, SB3, SB4, and SB5, showing that the transcription signatures of biosimilar sunscreen formulations form distinct clusters in unsupervised hierarchical cluster analysis of the transcription factor signatures of the sunscreens and their UV-blocking components.

The active ingredients of the respective formulations are identified in Table 8 below.

TABLE 8
Active Ingredients of the Sunblocks

| SB_1 | SB_2 | SB_3 | SB_4 | SB_5 |
|---|---|---|---|---|
| Avoenzone 3% | Homosalate 15%* | Avobenzone 3% | Titanium Dioxide 4.9% | Avobenzone 3% |
| Homosalate 13%* | Octinoxate 7.5% | Homosalate 10%* | Zinc Oxide 4.7% | Homosalate 15%* |
| Octisalate 5% | Octisalate 5% | Octisalate 5% | | Octisalate 5% |
| Octocrylene 7% | Titanium Dioxide 2.4% | Octocrylene 2.8% | | Octocrylene 4% |
| Oxybenzone 4% | | Oxybenzone 6% | | Ontenzone 5% |

The names of the ingredients of the formulations and their abbreviations in FIG. 15 are identified in Table 9 below.

TABLE 9

| Name of Ingredient: | Abbr. |
|---|---|
| 4-Aminobenzoic Acid | 4ABA |
| Avobenzone | ABZ |
| Sulisobenzone | BHZ |
| Oxybenzone | BP3 |
| Dioxybenzone | DMB |
| Octisalate | EHS |
| Octyl methoxycinnamate | EMC |
| Menthyl Anthranilate, 98% | MAN |
| Octocrylene | OCR |
| Phenylbenzimidazole sulfonic acid | PBA |
| padimate-O | PDO |
| Titanium Dioxide | TIOX |
| Zinc Oxide | ZnO |

The foregoing data and results show that the apparatus and methodology of the present disclosure enable the quantitative determination of biosimilarity in a simple, effective, and accurate manner.

Various aspects of the invention are further described below, in specific implementations and embodiments, as contemplated by the invention.

As discussed, the biosimilarity determination can be carried out in a method of determining biosimilarity of a sample composition to a reference composition, comprising:

exposing a test cell system to a sample composition so that the test cell system responds to the sample composition by change in transcription factor activity in said test cell system;

generating from the test cell system response an output correlative to the change of transcription factor activity in said test cell system; and

determining from comparison of said output with a transcription factor activity reference standard for the reference composition, the biosimilarity of the sample composition to the reference composition

In another embodiment, a method of determining biosimilarity of compositions is carried out, comprising:

exposing corresponding test cell systems to compositions to be assessed for biosimilarity;

generating profiles of ensuing changes in activities of transcription factors in said test cell systems in response to said exposing; and

determining from the generated profiles the presence or absence and degree of biological similarity of said compositions to one another.

In one embodiment of such method, a transcription factor signature is generated for each of the generated profiles, by constructing a library of reporter transcription units (RTUs), in which each RTU is constructed to include a common plasmid backbone and a unique transcription factor-inducible promoter that is fused to a transcribed reporter sequence, and transfecting the library of RTUs into cells of the test cell systems. The transfected library of RTUs can be arranged to produce reporter RNAs in amounts that are commensurate with the activities of the corresponding transcription factors present in the cells of the test cell systems. The method may further be conducted, wherein all RTUs are supplied with essentially identical reporter sequences.

In the above method, each reporter sequence can be advantageously tagged with a processing tag comprising a restriction cleavage site, the position of which varies among the RTUs. The operation of generating the profiles may comprise cleaving the restriction cleavage site to yield cleaved reporter species, and further comprise resolving the cleaved reporter species by capillary electrophoresis, in which the cleaved reporter species preferably are fluorescently labeled. The method may be conducted, in which the cleaved reporter species are produced from the reporter RNAs by reverse transcription and cDNA amplification by polymerase chain reaction using common pairs of primers for all reporter species. In a further embodiment, the method may also comprise generating the profiles in the form of polar coordinate radar graphs, and/or subjecting the generated profiles to spreadsheet analysis in said determining the presence or absence and degree of biological similarity of said compositions to one another.

In one embodiment of the present disclosure, the method may be conducted in which at least one of the steps of generating profiles of ensuing changes in activities of transcription factors in the test cell systems in response to the exposing, and determining from the generated profiles the presence or absence and degree of biological similarity of the compositions to one another, comprises a computer-implemented processing operation. In other embodiments, both of the generating and determining comprise computer-implemented processing operations.

In the conduct of capillary electorphoresis, data from the capillary electrophoresis is advantageously processed to algorithmically subtract background fluorescence for sizing of reporter peaks and noise/reporter peak discrimination, and fluorescence values of individual reporter peaks are desirably normalized to a sum of signals of all reporter peaks.

Methods of the disclosure can be variously carried out, wherein the transcription factors profiles comprise profiles of from 1 to 50 transcription factors. In a specific embodiment, the transcription factors profiles comprise profiles of from 2 to 10 transcription factors.

When the methods of the disclosure are carried out to generate respective transcription factor signatures, the respective transcription factor signatures are suitably algorithmically compared in said determining, in a computer-implemented determining process. In such method, the computer-implemented determining process may comprise use of an algorithmic technique selected from the group consisting of Pearson methods, Chebyshev correlation coefficients, Euclidean distance techniques, non-parametric methods, and cluster analysis techniques.

The methods of the disclosure can be conducted, wherein the test cell system comprises cells selected from the group consisting of individual cells, cell cultures, single-cell organisms, microbial populations, multicellular organisms, biological specimens taken or derived from such organisms, organs, tissue samples, tissue cultures, endogenous cells, exogenously modified cells, synthetic cells, human cells, animal cells, cloned cells, plant cells, blood cells, platelets, cultured cells, biopsied cells, cells fixed with preservatives, cells bound to substrates, nucleated cells, and non-nucleated cells.

In some embodiments of the method broadly described herein, the generating and determining operations may comprise assessing changes in DNA-binding activities in cell extracts, e.g., comprising use of a gel-shift assay. Alternatively, the generating and determining operations may comprise analyzing changes in cellular localization of transcription factors, such as nuclear translocation of transcription factors.

In still other embodiments, the generating operation can comprise representing transcription factor activity profiles by vectors with coordinates x1, x2 . . . xN, where xi is the activity of the i^{th} transcription factor, TFᵢ, and such determining comprises assessing Euclidian distance between transcription factor activity vectors.

In various embodiments of the method of the disclosure, the compositions can be selected from the group consisting of compounds, chemical complexes, elements, ionic species, multi-component formulations, cells, ligands, microbes, nucleic acids, proteins, peptides, antigens, receptors, antibodies, nutrients, minerals, environmental samples, and components, fragments and/or contaminants of the foregoing.

Another aspect of the disclosure relates to a method of determining biosimilarity of compounds, comprising quantifying impacts of said compounds on activities of multiple transcription factors in a test cell system.

A further aspect of the disclosure relates to a method of determining biosimilarity of different compositions, comprising:

exposing each different composition to a corresponding biosensor comprising multiple transcription factors, wherein the corresponding biosensor is adapted to manifest a transcription factor signature in response to the exposure; and

comparing transcription factor signatures of the corresponding biosensors, or of their expression products, to determine biosimilarity of the different compositions in relation to one another.

Another aspect of the disclosure relates to a method of determining biosimilarity of different compositions, comprising:

introducing into a test cell system comprising a multiplicity of transcription factors, a plurality of reporter constructs whose promotors are regulated by the transcription factors;

exposing the test cell system to the different compositions to induce corresponding changes in activities of said multiplicity of transcription factors; and

determining biosimilarity of the different compositions from a plurality of reporter transcripts produced by the reporter constructs and/or a plurality of reporter proteins produced by the reporter constructs in response to the changes in activities of said multiplicity of transcription factors upon exposing the test cell system to the different compositions.

The disclosure in a further aspect relates to an apparatus for determining biosimilarity of different compositions, comprising a computer system adapted to carry out an operation of a method according to any of embodiments above described herein. The apparatus may be configured with the computer system comprising networked computers, e.g., a central server computer and a client computer.

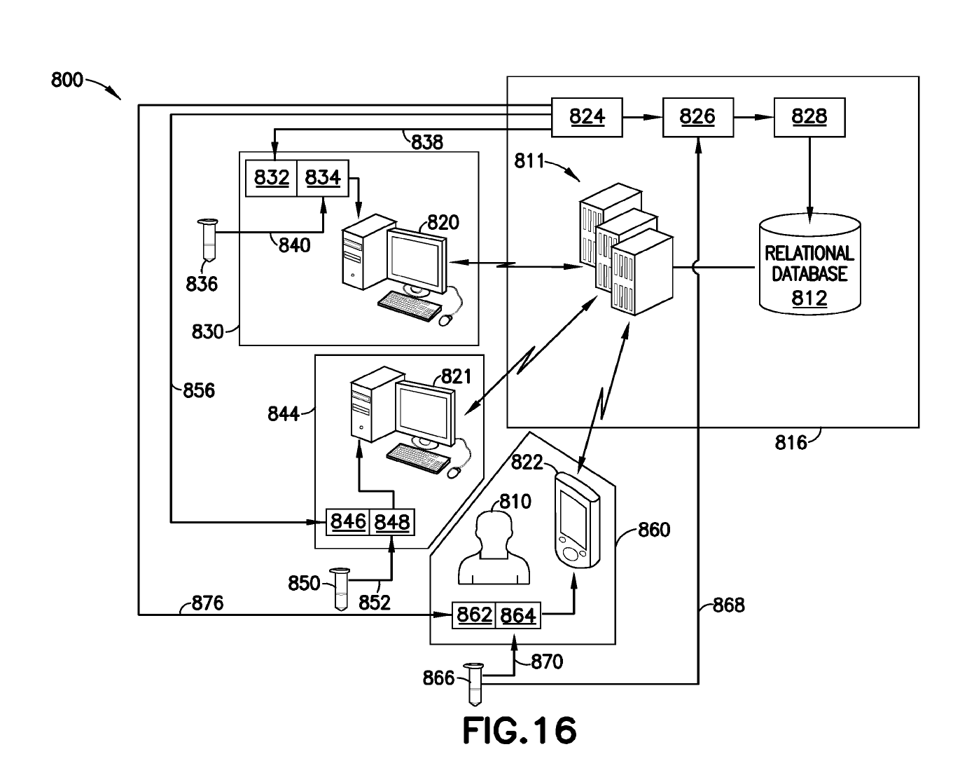

FIG. 16 is a schematic representation of a biosimilarity assessment system, including a central water quality administration facility, arranged for sample handling and storage, biosensor (test cell) cell culturing, cell plating and processing, database and data analysis, and linked in communication relationship with remote sample input and processing facilities, which may be optionally supplied with reagents, biosensor (test cell) products, and process equipment support from the central facility.

The biosimilarity assessment system 800 includes a central biosimilarity assessment administration facility 816, in which is disposed a server assembly 811 comprising multiple server units operatively linked to a relational database 812 that may for example contain a library of transcription factor activity reference standards for reference compositions, as well as protocols for conducting biosimilarity assessment assays using the transcription factor activity methodology of the present disclosure, and other data, accessible to the server units for computational and communicational operations.

The biosimilarity assessment administration facility 816 also includes a storage inventory of supplies 824, for conducting biosimilarity assessment assays in accordance with the present disclosure, including reverse transcription, PCR, and fluorescent labeling reagents, capillary electrophoresis equipment and supplies, biosensor (test cell) units, cell plating equipment and supplies, computational devices adapted for use at remote sample input and processing facilities, sample collection and storage apparatus, etc.

The central biosimilarity assessment administration facility 816 further includes a sample processing unit 826 in which a sample can be contacted with the biosensor (test cells), following which the test cells can be submitted to total RNA isolation, reverse transcription, PCR amplification, fluorescent labeling, restriction digestion, sample clean-up, and capillary electrophoresis. The central facility 816 also includes a primary data analysis unit 828 arranged to receive output from the processing unit 826 and to generate profiles therefor, e.g., for reference samples, or for samples to be assayed as received from a remote sample input and processing facility 860, as hereinafter more fully described.

The system 800 illustratively shown in FIG. 16 includes multiple remote sample input and processing facilities 830, 844, and 860. Each of the remote sample input and processing facilities may be located at substantial distances from the central facility, e.g., in different cites or countries, or even different continents. The remote sample input and processing facilities can be variously constituted, but each is coupled in communication relationship with the central biosimilarity assessment administration facility 816 for bidirectional transmission and receipt of data and signal communications. Such communication coupling may comprise interconnection via a worldwide communication network such as the internet, or other network.

The remote sample input and processing facility 830 is arranged for processing of local samples 836 that may be introduced to the remote facility as schematically indicated by arrow 840, and assayed in accordance with the transcription factor activity methods of the present disclosure, in processing module 834 which is supplied with equipment and supplies from the supply module 832. The supply module 832 in turn may be supplied from the storage inventory of supplies 824 from the central facility 816, as schematically indicated by arrow 838.

Local sample assay data generated by the processing module 834 may be transmitted to the central processor unit (CPU) 820 for further processing and transmission to the servers 811 of the central facility 816. The servers 811 then may effect an algorithmic comparison of a transcription factor activity profile for the sample 836 with a reference standard transcription factor activity profile, and provide resulting biosimilarity comparison data back to the central processor unit (CPU) 820 for local usage at the remote sample input and processing facility 830.

The remote sample input and processing facility 844 is likewise arranged for collection and processing of local samples 850 that may be introduced to the remote facility as schematically indicated by arrow 852, and assayed in accordance with the transcription factor activity methods of the present disclosure, in processing module 848 which is supplied with equipment and supplies from the supply module 846. The supply module 846 in turn may be supplied from the storage inventory of supplies 824 from the central facility 816, as schematically indicated by arrow 856.

Local sample assay data generated by the processing module 848 may be transmitted to the central processor unit (CPU) 821 for further processing and transmission to the servers 811 of the central facility 816. The servers 811 then may effect an algorithmic comparison of a transcription factor activity profile for the sample 850 with a reference standard transcription factor activity profile, and provide resulting biosimilarity comparison data back to the central processor unit (CPU) 821 for local usage at the remote sample input and processing facility 844.

The remote sample input and processing facility 860 includes a supply module 862 that can be supplied with equipment, reagents, and other supplies from the storage inventory of supplies 824 from the central facility 816, as schematically indicated by arrow 876. The supplies from the local supply module 862 are used in processing module 864, which receives local sample 866, as schematically indicated by arrow 870. In the processing module 864, a local sample assay is conducted in accordance with the transcription factor activity methodology of the present disclosure, with resulting data being passed to the smartphone 822 of user 810 at the remote facility, and then transmitted to the servers 811 of the central facility, for referential comparison of the transcription factor activity profiles generated at the remote facility 860, to generate a biosimilarity assessment output that is transmitted back to the smartphone, for real-time determinations of the biosimilarity of the local sample to reference standard(s).

Alternatively, the local sample 866 can be transmitted directly by the remote facility 860 to the central facility 816, as schematically indicated by arrow 868.

The central facility 816 can also be arranged to provide technical support to the remote facilities, e.g., with updated algorithms, protocols, regulatory updates, etc., as communicated by server units 811 to the remote facilities.

In a further aspect, the disclosure relates to a kit for carrying out biosimilarity determinations of compositions, comprising transcription factor signatures for reference library compositions in a graphical format, for threshold visual determinations of biosimilarity of a transcription factor signature to reference library signatures.

Another aspect of the disclosure relates to a kit for carrying out biosimilarity determinations of compositions, comprising biosensors, contacting containers in which cells may be contacted with compositions of interest, and instructional documents containing protocols for conducting the contacting operation, and the further processing of the contacted cell samples for analysis of transcription factor signatures. Such kit may further comprise one or more of lyzing media, transfection vectors, restriction enzymes, reverse transcription reagents, PCR primers, fluorescent dyes, discs or flash drives containing capillary electrophoresis data processing software, transcription factor signature-generation software, and/or software for conducting other component operations in the biosimilarity determinations.

While the disclosure has been has been set forth herein with reference to specific aspects, features and illustrative embodiments, it will be appreciated that the utility of the disclosure is not thus limited, but rather extends to and encompasses numerous other variations, modifications and alternative embodiments, as will suggest themselves to those of ordinary skill in the field of the present disclosure, based on the description herein. Correspondingly, the invention as hereinafter claimed is intended to be broadly construed and interpreted, as including all such variations, modifications and alternative embodiments, within its spirit and scope.

1. A method of determining biosimilarity of a sample composition to a reference composition, comprising:
exposing a test cell system to a sample composition so that the test cell system responds to the sample composition by change in transcription factor activity in said test cell system;
generating from the test cell system response an output correlative to the change of transcription factor activity in said test cell system; and
determining from comparison of said output with a transcription factor activity reference standard for the reference composition, the biosimilarity of the sample composition to the reference composition,
wherein the method comprises at least one of:
(i) generating a transcription factor signature for the output correlative to the change of transcription factor activity in said test cell system, by constructing a library of reporter transcription units (RTUs), in which each RTU is constructed to include a common plasmid backbone and a unique transcription factor-inducible promoter that is fused to a transcribed reporter sequence, and transfecting the library of RTUs into cells of the test cell system; and
(ii) representing transcription factor activity profiles by vectors with coordinates x1, x2 . . . xN, where xi is the activity of the i^{th} transcription factor, TFᵢ, and assessing Euclidian distance between transcription factor activity vectors.

2. The method of claim 1, wherein the transcription factor activity reference standard for the reference composition comprises an averaged transcription factor activity reference for multiple samples of the reference composition.

3. (canceled)

4. The method of claim 1, wherein the test cell system comprises a promoter in reporter transcription units that is responsive to multiple transcription factors.

5. The method of claim 1, wherein the test cell system comprises a panel of cells of differing types, wherein each of said cell types responds to exposure to the sample composition by change of transcription factor activity of only one transcription factor that is different in each of the cell types, or by change of transcription factor activity of multiple transcription factors that are different in each of these cell types.

6. (canceled)

7. (canceled)

8. The method of claim 1, wherein the transfected library of RTUs produce reporter RNAs in amounts that are commensurate with the activities of the corresponding transcription factors present in the cells of the test cell systems.

9. The method of claim 8, wherein all RTUs are supplied with essentially identical reporter sequences, wherein each reporter sequence is tagged with a processing tag comprising a restriction cleavage site, the position of which varies among the RTUs, wherein said generating said profiles comprises cleaving the restriction cleavage site to yield cleaved reporter species, further comprising resolving the cleaved reporter species by capillary electrophoresis, wherein the cleaved reporter species are fluorescently labeled, and wherein the cleaved reporter species are produced from the reporter RNAs by reverse transcription and cDNA amplification by polymerase chain reaction using common pairs of primers for all reporter species.

10. (canceled)

11. (canceled)

12. (canceled)

13. (canceled)

14. (canceled)

15. The method of claim 1, wherein said output is in the form of polar coordinate radar graphs.

16. The method of claim 14, further comprising spreadsheet analysis in said determining, of the presence or absence and degree of biological similarity of said compositions to one another.

17. The method of claim 1, wherein at least one of said generating and said determining comprises a computer-implemented processing operation.

18. (canceled)

19. The method of claim 9, wherein data from the capillary electrophoresis is processed to algorithmically subtract background fluorescence for sizing of reporter peaks and noise/reporter peak discrimination, and wherein fluorescence values of individual reporter peaks are normalized to a sum of signals of all reporter peaks.

20. (canceled)

21. The method of claim 1, wherein the output comprises transcription factors profiles of from 1 to 50 transcription factors.

22. (canceled)

23. The method of claim 1, wherein transcription factor signatures are algorithmically compared in said determining, in a computer-implemented determining process.

24. The method of claim 23, wherein the computer-implemented determining process comprises use of an algorithmic technique selected from the group consisting of Pearson methods, Chebyshev correlation coefficients, Euclidean distance techniques, non-parametric methods, and cluster analysis techniques.

25. The method of claim 1, wherein the test cell system comprises cells selected from the group consisting of individual cells, cell cultures, single-cell organisms, microbial populations, multicellular organisms, biological specimens taken or derived from such organisms, organs, tissue samples, tissue cultures, endogenous cells, exogenously modified cells, synthetic cells, human cells, animal cells, cloned cells, plant cells, blood cells, platelets, cultured cells, biopsied cells, cells fixed with preservatives, cells bound to substrates, nucleated cells, and non-nucleated cells.

26. The method of claim 1, wherein said generating and determining comprise assessing changes in DNA-binding activities in cell extracts.

27. (canceled)

28. The method of claim 1, wherein said generating and determining comprise analyzing changes in cellular localization of transcription factors.

29. (canceled)

30. The method of claim 1, wherein the generating comprises representing transcription factor activity profiles by vectors with coordinates x1, x2 . . . xN, where xi is the activity of the i^{th} transcription factor, TFᵢ, and said determining comprises assessing Euclidian distance between transcription factor activity vectors

31. The method of claim 1, wherein the sample composition is selected from the group consisting of compounds, chemical complexes, elements, ionic species, multi-component formulations, cells, ligands, microbes, nucleic acids, proteins, peptides, antigens, receptors, antibodies, nutrients, minerals, environmental samples, and components, fragments and/or contaminants of the foregoing.

32. (canceled)

33. (canceled)

34. (canceled)

35. An apparatus for determining biosimilarity of different compositions, comprising a computer system adapted to carry out an operation of a method according to claim 1.

36. (canceled)

37. (canceled)

38. (canceled)

39. A kit for carrying out biosimilarity determinations of compositions, comprising at least one of (i) and (ii):
(i) transcription factor signatures for reference library compositions in a graphical format, for threshold visual determinations of biosimilarity of a transcription factor signature to reference library signatures; and
(ii) biosensors, contacting containers in which cells may be contacted with compositions of interest, and instructional documents containing protocols for conducting the contacting operation, and the further processing of the contacted cell samples for analysis of transcription factor signatures, optionally further comprising one or more of lyzing media, transfection vectors, restriction enzymes, reverse transcription reagents, PCR primers, fluorescent dyes, discs or flash drives containing capillary electrophoresis data processing software, transcription factor signature-generation software, and/or software for conducting other component operations in the biosimilarity determinations.

40. (canceled)

41. (canceled)
